(19) United States
(12) Patent Application Publication
(10) Pub. No.: US 2016/0199148 A1
(43) Pub. Date: Jul. 14, 2016

(54) ENDO-NAVIGATION SYSTEMS AND METHODS FOR SURGICAL PROCEDURES AND CPR

(71) Applicant: Luiz MARACAJA-NETO, San Antonio, TX (US)

(72) Inventor: Luiz MARACAJA-NETO, San Antonio, TX (US)

(73) Assignee: THE BOARD OF REGENTS OF THE UNIVERSITY OF TEXAS SYSTEM, Austin, TX (US)

(21) Appl. No.: 14/914,720

(22) PCT Filed: Sep. 1, 2014
(86) PCT No.: PCT/US14/53630

§ 371 Date: Feb. 26, 2016

Related U.S. Application Data
(60) Provisional application No. 61/872,124, filed on Aug. 30, 2013.

(51) Int. Cl. A61B 90/00; A61B 1/04

(52) U.S. Cl. CPC A61B 90/39; A61B 1/04; A61B 2034/2055

(57) ABSTRACT

Certain embodiments are directed to an image-based optical tracking and navigation system. The system being useful during surgical procedures or cardiopulmonary resuscitation. The navigation system combining external optical tracking of instrument handles or shafts and positional endoscope tracking techniques with images of the patient's internal structures. The imaging instrument can be one of the following: an endoscope, a colonoscope or a transephageal echo probe. Magnetic probes/stylets can also be used to further determine the distal end of the imaging instrument.

This application claims priority to U.S. Provisional Patent Application No. 61/872,124 filed Aug. 30, 2013, which is incorporated herein by reference in its entirety.

### BACKGROUND

I. Field of the Invention

Certain embodiments are in the general field of medicine and endoscopy, in particular devices, systems, and methods for use of positional imaging navigation systems for guidance of surgical procedures.

II. Background

Current surgical navigation uses imaging and computer technology for presurgical planning to guide surgical interventions. Before surgery, medical imaging techniques are used to create a 3D data set model of the anatomy. This model may then be used by a navigation system to reproduce the spatial relationship between the normal tissues, the pathologic tissues, and surgical instruments. This technology was initially introduced in neurosurgery for resection of brain tumors and quickly became available for other medical specialties.

In certain surgical navigation methods fiducial markers can be placed at specific anatomic regions during diagnostic scanning, which can be done by computer tomography (CT), ultrasound, or magnetic resonance imaging (MRI) scan. The images obtained are uploaded to a computer navigation system where image analysis and manipulation are performed using contrast techniques that can differentiate the tissues and extract relevant information from the data set to produce an anatomic model. Fiducial markers can also be affixed during surgery to the same anatomic regions used during the diagnostic scan to guide the surgery or surgeon. The computer navigation system may be configured to combine the imaging data with the positions of the fiducial markers. Computer software from the navigation system renders virtual 2D or even 3D images from the image data set in relationship with the surgical instruments. The view on the display can be manipulated to provide views with different depth and angles of view.

Surgical navigation allows physicians to visualize in real time the desired anatomic structure and trajectory of the surgical instruments. It allows the surgeon to plan the incision before cutting the skin, starting close to the desired anatomic structure and avoiding accidental lesion or cut of healthy structures. Using custom software allows the surgical intervention to be planned and simulated virtually. Furthermore, the data can be used to program a robot to perform pre-defined maneuvers during the surgery.

As opposed to static organs, the heart and lungs are examples of dynamic organs that are constantly moving in order to maintain their vital functions. During a normal cardiac cycle, the heart contracts during systole and relaxes during diastole; the lungs expand during inspiration and recoil during the expiration. Changes of volume and shape occur in the heart depending on the amount of blood necessary to be pumped; and in the lungs depending on the volume of gas inspired. Changes of pressure in the pleural and pericardial cavity change with position of the heart (e.g. positive pressure ventilation, pleural effusion, pneumothorax, lung hyperinflation or collapse). The forces of gravity, changes in body position, and tissue swelling also affect the position of the heart and lungs in the chest and may affect the shape of the lungs. The lungs are normally very complacent and easily compressible by external pressures exerted by the chest wall, diaphragm, and abdominal contents including liver or bowel loops. The lung segments can also collapse during a process known as atelectasis causing changes in their shape.

Because of the above-cited reasons, several circumstances frequently occur during the course of a surgical procedure under general anesthesia, such as positional changes, positive pressure ventilation, blood volume variation—which may cause a distortion to the accuracy of the preplanned procedure due to the shift of tissues in comparison to their original position during the pre-surgical imaging and planning

The acquisition of the data set with the currently available imaging systems are not able to detect the above-cited changes making the spatial orientation inaccurate. Thus, there is a need for additional systems and devices for imaging non-static organ (dynamic organs) during surgery.

### SUMMARY

Embodiments are directed to surgical imaging and navigation systems that can be used during internal surgery, during cardiopulmonary resuscitation, and for various cardiothoracic procedures including open-heart surgery, minimally invasive surgery, endoscopic, robotic, and catheter based interventions. In certain aspects the imaging systems described herein can be used in surgical procedures involving intricate winding or branched internal anatomy such as tracheal bronchial tree, gastro-intestinal tract, and bile-pancreatic tract. The systems, devices, and procedures described herein can be used to improve visualization during surgery and optimize results.

Certain embodiments are directed to an imaging guide navigation system comprising catheter(s), endoscope(s), and/or instrument(s) optically marked and a camera system to capture and process images of the marked instruments and integrate this information with imaging of a subject. An endoscope is an instrument used to examine the interior of a hollow organ or cavity of the body. In certain aspects an endoscope comprises a rigid or flexible tube, a light delivery system to illuminate the organ or object under inspection (the light source is normally outside the body and the light is typically directed via an optical fiber system); a lens system transmitting the image from the objective lens to the viewer, typically a relay lens system in the case of rigid endoscopes or a bundle of fiberoptics in the case of a fiberscope; an eyepiece (instruments may be videoscopes with no eyepiece, a camera transmits image to a screen for image capture; and an additional channel to allow entry of medical instruments or manipulators. Optical marking uses external optical tracking (optical fiducial(s) are visible from outside a body) supplemented with positioning data obtained from an imaging device. Imaging can be done by sonogram, x-ray, magnetic resonance, etc. imaging can be accomplished through an endoscope or by extracorporeal methods. In certain aspects an imaging sensor or detector is position at the tip of an endoscope that is deployed to assist or perform portions of the surgery.

The camera system is a computerized external camera-based optical tracking system utilizing a plurality of optical fiducials as referencing markers attached to or built into an endoscope or surgical instrument. In certain aspect the scope is a transesophageal echocardiography (TEE) ultrasound transducer. In further aspects the surgical instruments are instruments designed to traverse the skin or body wall, such as laparoscopic instruments including but not limited to scissors, graspers, dissectors, electrodes, suturing devices, trocars, cameras, and the like.

A fiducial marker or fiducial is an object placed in the field of view of an imaging system which appears in the image produced, for use as a point of reference or a measure. It may be either something placed into or on the imaging subject. The optical fiducial(s) used in conjunction with the medical procedures described herein are markers that can be distinguish from one another and from which the camera system can triangulate a position. The optical fiducials are designed to be able to convey a change in position, translation and/or rotation, when the instrument they are associated with is moved or adjusted. The relationship between a fiducial or a set of fiducials and an instrument, catheter, or endoscope can be programmed into or determined by the camera system. As such the external optical fiducial provides an external indicator of the position of an instrument relative to an internal organ or body part being imaged. The fiducial can be of any design that meets the needs of the system. Some examples of a fiducial include a multi-pronged device having termini with distinguishing features, such as size, shape, color, etc. so that each individual fiducial of set of fiducials can be identified and tracked by the camera system. The fiducials described herein will be of a material and/or color that can be easily captured and tracked by the camera system when in a surgical or medical environment. In certain aspects the termini are spheres, triangles, cubes, or other three dimensional polygons. The arms of the fiducial can be set a predetermined angle with respect to other arms of the fiducial. In certain aspects a fiducial, arm, or termini can have reference marks identifiable by the camera system.

The camera system is configured to track the spatial coordinates of the optical fiducial markers independently to obtain reference points and distance between the markers. Optical external markers or landmarks may also be built in or attached onto other elements of the CPR equipment or surgical instruments.

A navigation system described herein can be used in manipulating an endoscope. The navigation system can be integrated with other sensors incorporated into an endoscope or other instruments to provide greater positional resolution. For example, an endoscope positioning technology can comprise a semi-flexible stylet in which the deformation of the stylet material can be measured and translated to the system to account for various curvatures and bends traversed by the endoscope or instrument. In certain aspects the endoscope is a transesophageal echocardiography (TEE) ultrasound transducer.

Portions of an endoscope (e.g., the tip of the scope) or other instrument can also be located using site emission sensors for electromagnetic signals, such as radio waves, emitted from a portion of the endoscope or other instrument. These waves can be detected outside of the patient and provide a reference for positioning a scope or instrument. The navigation or tracking system may be able to map the spatial relationship between the distal end of a probe (e.g., TEE transducer) through external reference points or markers attached to the proximal end of the probe and further tie them together or integrate the position with CPR or surgical instruments. An endoscope or instrument utilizing site emission technology can be used in conjunction with external optical markers designed for that purpose.

A tracking system may be calibrated with a specific size and type of endoscope or instrument in order to determine the exact location of the endoscope or instrument. For example, the tracking system is calibrated with a specific size and type of TEE in order to determine the exact location of the ultrasound source. In certain aspects the ultrasound source is a small area located at about the distal tip of the transducer and is used as initial reference tracking point. The reference point can be selected or altered according to the desired cardiac structure imaged with the echocardiography.

Also provided is novel software program configured to tie together or integrate spatial location of the TEE probe and CPR or surgical tools as well as data acquired from real time 2D or 3D echocardiography images. The system can also use other imaging technologies such as MRI or CT scan.

The computerized display allows the real time visualization of the reference point “heart or specific structure.” The visualized structure is then displayed in relationship with CPR or surgical instruments. This system provides spatial accuracy of internal dynamic structures (e.g., heart structures) and precise spatial guidance of the operative procedure and more efficient performance of CPR, for example.

Certain embodiments are directed to the use of flexible endoscope-like and steerable catheter devices associated with positional imaging navigation system for guidance of surgical procedures. Endoscopes include steerable devices equipped with fiber optic, video cameras and endoscopic ultrasound imaging probes. Certain embodiments are directed to a cardiothoracic navigation system for providing accurate spatial location of cardiothoracic structures and guide manipulation of surgical instruments during surgical operations or devices/apparatus employed for CPR. An optical stylet with electromagnetic elements and magnetometers may be used to precisely determine the curvatures of the tip portion of an endoscope, e.g., the tip of a TEE probe. The computer unit of the system may be configured to receive the patient's imaging data set acquired using CT, MRI, or echocardiography; and also receive data from external optical and electromagnetic position tracking Software may be used to provide for combining of the imaging data set with the positional tracking data.

A computer terminal with screen display may be used to provide rendered virtual images with 2D or 3D views for different depths and viewing angles, and define the positional relationship of instruments used during surgery or CPR.

The system of the invention may also be configured to import other patient visualization data such as MRI or CT scan.

The computerized display may be configured to allow real time visualization of the reference markers as well as heart or specific internal structures of interest and their spatial relationship with the CPR or surgical instruments. This system provides spatially accurate representation of the heart structures and precise guidance of the operative procedure or more efficient performance of CPR.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIG. 1 illustrates a transesophageal echocardiography transducer with optical fiducial markers inserted in different parts of the transducer.

FIG. 2 shows cardiac navigation computer unit with graphic display and a stand unit with two cameras mounted thereon.

FIG. 3. (A) Shows a TEE probe inserted into a patient, with the insert. (B) Showing a view of the heart as provided by the TEE transducer.

FIG. 4 illustrates various fiducial optical markers attached to command knobs of the TEE probe and the respective movement of the distal tip of the transducer.

FIG. 5 illustrates tracking curvature mechanism embodied within the TEE transducer.

FIG. 6 illustrates a schematic diagram of surgical instruments with optical marker inserted into chest cavity during a minimally invasive surgery.

FIG. 7 (A) Illustrates different types of surgical instruments with different handles equipped with optical markers. (B) Shows different types of needles, guidewires and percutaneous sheaths that may be used for the purposes of the present invention.

FIG. 8 illustrates hand-operated chest compression with a hand attachment containing external optical markers.

FIG. 9 illustrates a CPR device to be positioned on the chest for manual chest compression—equipped with optical markers.

FIG. 10 illustrates a CPR device to be positioned around the chest for mechanical chest compression—equipped with optical markers.

FIG. 11 illustrates a defibrillation handle equipped with optical markers.

FIG. 12 illustrates adhesive pads for defibrillation and heart pacing—equipped with optical markers of the invention.

FIG. 13 shows a colonoscopy surgical procedure in which a colonoscope is equipped with the external optical markers of the invention.

FIG. 14 shows an endoscopy procedure, in which the distal part of the endoscope is illuminating a specific part of a gastrointestinal tract. Using the present invention may improve outcomes of such procedures.

FIG. 15 shows another endoscopy procedure, in which the distal part of the endoscope is illuminating a specific part of the biliary and pancreatic tract. Performing the endoscopic procedure using the system of the invention may improve clinical outcomes of such procedures.

### DESCRIPTION

The devices, apparatus, and systems described herein overcome the above described limitations of the current systems. Embodiments are directed to a cardiac navigation system that receives imaging and positional data set in real time from a TEE transducer with optical and magnetic reference markers. This process allows acquisition and frequent recalibration of the reference points and accounts for all of the distortions associated with a dynamic organ.

Spatial reference data may be promptly generated with the insertion of a TEE probe. In certain aspects a TEE probe can be used for clinical situations such as emergency surgery or cardiopulmonary resuscitation, where no time is available to perform CT or MRI scan. One such emergency situation is cardiac arrest, which is the cessation of normal blood circulation due to failure of the heart to contract effectively. The lack of blood perfusion prevents delivery of the nutrients and oxygen to the tissues. Lack of oxygen is very critical and poorly tolerated by the brain and myocardium. Brain injury is likely to happen if cardiac arrest goes untreated even for a short period of time. Cardiopulmonary resuscitation (CPR) is an emergency procedure, performed in an effort to manually or mechanically assist blood circulation to preserve intact brain function until further measures are taken to restore spontaneous blood circulation.

Stroke volume is the volume of blood ejected by the left ventricle into the aorta in each cardiac cycle. In physiologic spontaneous circulation conditions during the systole, the walls of the left ventricle contract inward in a synchronized manner promoting increase of intraventricular pressure, which closes the mitral valve and opens the aortic valve. During chest compression in CPR, the stroke volume is produced by cardiac compression promoting increase of the intracavitary pressure and ejection of blood to maintain the circulation. Investigations related to chest compressions demonstrated that the stroke volume produced during chest compression is only 35% when comparing to the physiologically normal stroke volume. During CPR, the compression of the heart is performed between the sternum and spine or posterior mediastinum structures. The anterior to posterior compression promotes distortion and asynchrony of the ventricle walls and distortion of the mitral annulus resulting in mitral regurgitation.

Another important concept during CPR is point of maximal compression (PMC). The PMC is the part of the heart most intensely compressed during the chest compression. The PMC should ideally not be on top of the aorta or the left ventricle output tract. According to the biotype of the patient, there is also important variation on the heart positioning inside the chest; tall patients tend to have the heart positioned more vertically while short patients tend to have the heart positioned more horizontally.

Scientific investigations showed the PMC generated by traditional chest compressions is often located on top of the aorta and left ventricle output tract, thereby obstructing the blood flow. Recent medical literature indicates important benefits of using echocardiography during CPR. Particular modality of sonography called transesophageal echocardiography (TEE) where an ultrasound transducer is mounted at the tip of a flexible endoscope can be used during this procedure to enhance visualization and optimize CPR.

The main advantages of using TEE during CPR as cited in the medical literature are: it allows visualization of cardiac structures without interfering with the chest compressions; it allows for diagnosing new and/or unexpected conditions such as cardiac tamponade, pulmonary embolism, air-embolism, massive myocardium infarction, severe hypovolemia, aortic dissection; it can be used to detect the most efficacious location of chest compressions; it allows detection of fine ventricular fibrillation pattern, which could be interpreted as asystole by the electrocardiography; and it allows for a major improvement in clinical management and making subsequent medical decisions. The efficacy of CPR performance and maintenance of brain perfusion during cardiac arrest is critical for the clinical prognosis of the patient. The cardiac image-guided navigation during CPR provides for appropriate adjustments for CPR performance with real time visualization of the best possible positioning of optically tracked CPR apparatus or manual compression. A fiducial can be coupled to a compression piston or hand and the position determined and integrated with an echocardiograph to optimize position and depth of compression.

In regard to surgery, the minimally invasive surgery (MIS) techniques, with or without the use of robots provides several advantages when compared to the traditional open procedures. The benefits cited on the medical literature include: increased accuracy, because MIS procedures use image-assisted equipment, allowing the surgeon to make a more precise diagnosis and to obtain better visualization of desired anatomic structures; less tissue injury by using smaller incisions where the surgeon does not have to cut through a lot of tissue (Traditional open surgeries require big incisions causing lesions of several tissues such as skin, subcutaneous fat, muscles, and bone. In traditional open-heart surgery, the surgeon makes a ten- to twelve-inch long incision, then gains access to the heart by splitting the sternum and spreading open the rib cage. This large incision may cause higher rate of postoperative infection and respiratory dysfunction, and prolonged time to complete recovery. The damaged tissues need a significant time to heal after surgery); less post-operative pain and smaller consumption of pain medications, which has undesirable side effects; shorter hospital stay with quicker return to normal activities; smaller scars; lower rate of post-operative infections; and less blood loss.

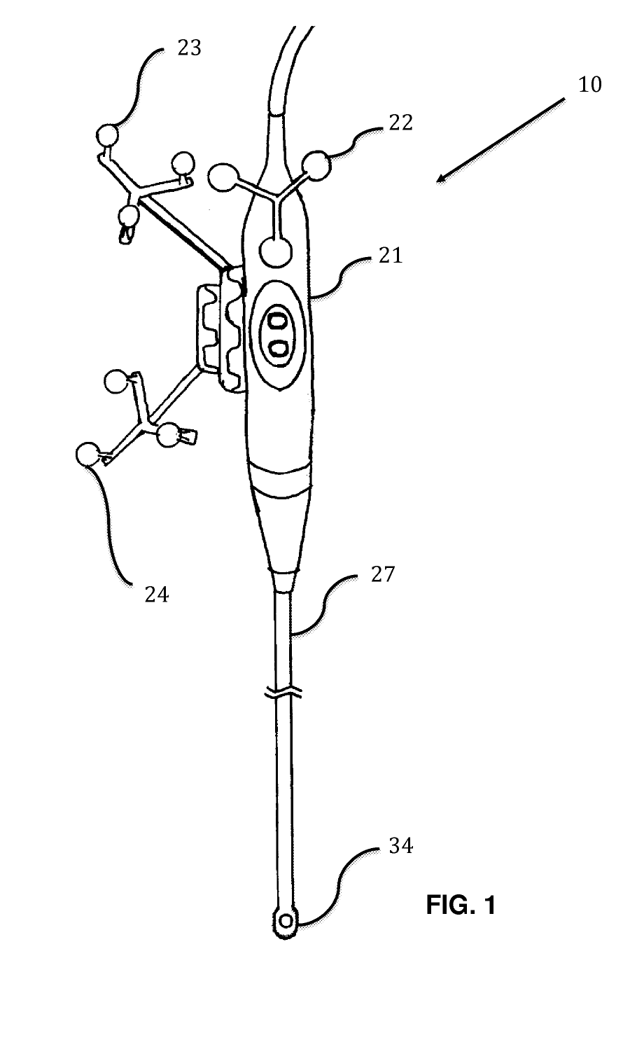

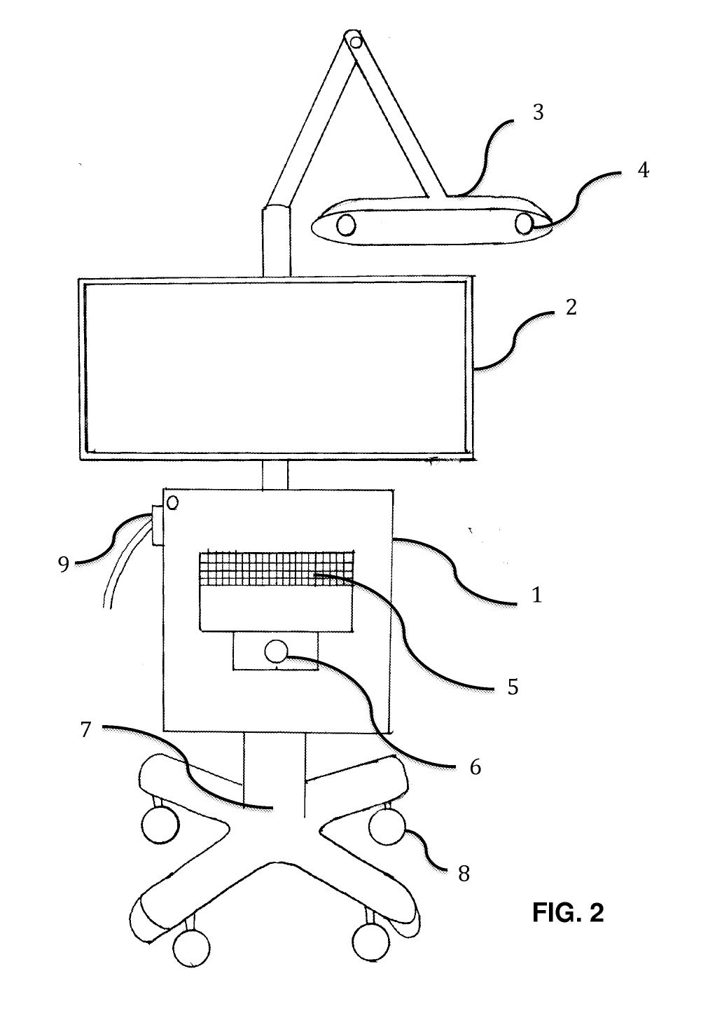

As described above, cardiac imaging using transesophageal echocardiography (TEE) can be used for diagnosis and monitoring of heart structures function. The TEE imaging probe 10 may include a handle 21, shaft 27, and a piezoelectric transducer 34. The fiducial markers with variable size, color, and shape may be attached to or built at various locations of the TEE probe 10. In certain aspects surgical tools can be distinguished during the procedure by associating balls of different size and/or color for each of the instruments. To further improve the ability of the system to detect location as well as motion of each instrument in 6 degrees of freedom, each marker may include three spaced apart portions, e.g., balls as seen in the figures. This design makes visible not only linear motion of the handle in any of the three coordinates X, Y, and Z, but also its rotation about any of the three orthogonal axes. Variations in desired position can occur depending on specific transducer design used, however for the purpose of this description the markers are represented as follows in FIG. 1: marker 22 is shown attached to the handle 21 of the probe 10; marker 23 is attached to the larger steering knob of the probe 10; marker 24 is attached to the smaller steering knob of the probe 10.

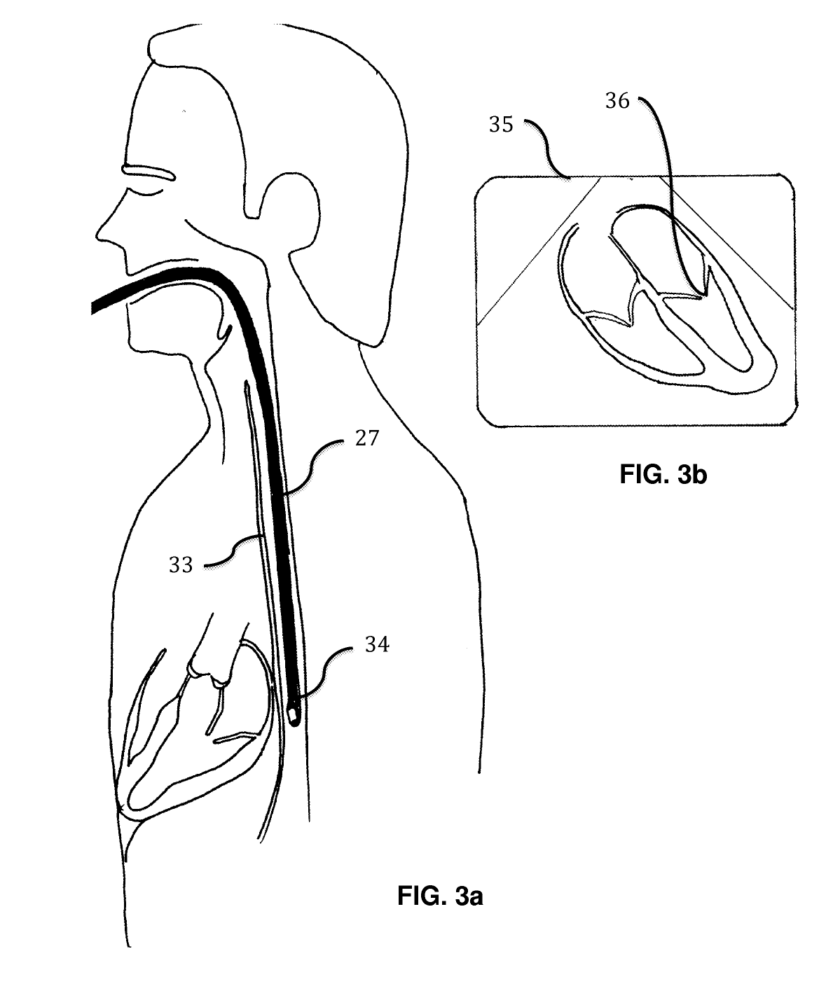

The navigation system of the invention may comprise a computer system 1 as seen in FIG. 2. Connected to the computer system 1 is a monitor display 2, an optionally adjustable and detachable mount position tracking system 3 with suitable position tracking cameras 4. Two or three cameras 4 may be used for the purposes of tracking the optical markers of the invention. Peripheral input components such as keyboard 5 and mouse 6 may also be provided along with the input jack 9 for the imaging data. Various external sources of imaging data may be used, including but not limited to a CT scan, MRI, or ultrasound sources, including 2D and 3D transesophageal echocardiography.

The entire system may be positioned on a wall or mounted on a movable cart 7 containing wheels 8 with locking mechanisms. The system may therefore be easily transported for use in various hospital locations such as Operating Room, Intensive Care Unit, and Emergency Department. The system of the invention may also be possibly located in ambulances for pre hospital use.

In use, the system 1 may be configured to detect the spatial location of the optical markers 22-24. Knowing the physical dimensions of the TEE probe 10 makes it possible to determine the spatial coordinates of the TEE transducer 34, which in turn may be used to assign specific coordinates to the image provided by the transducer 34.

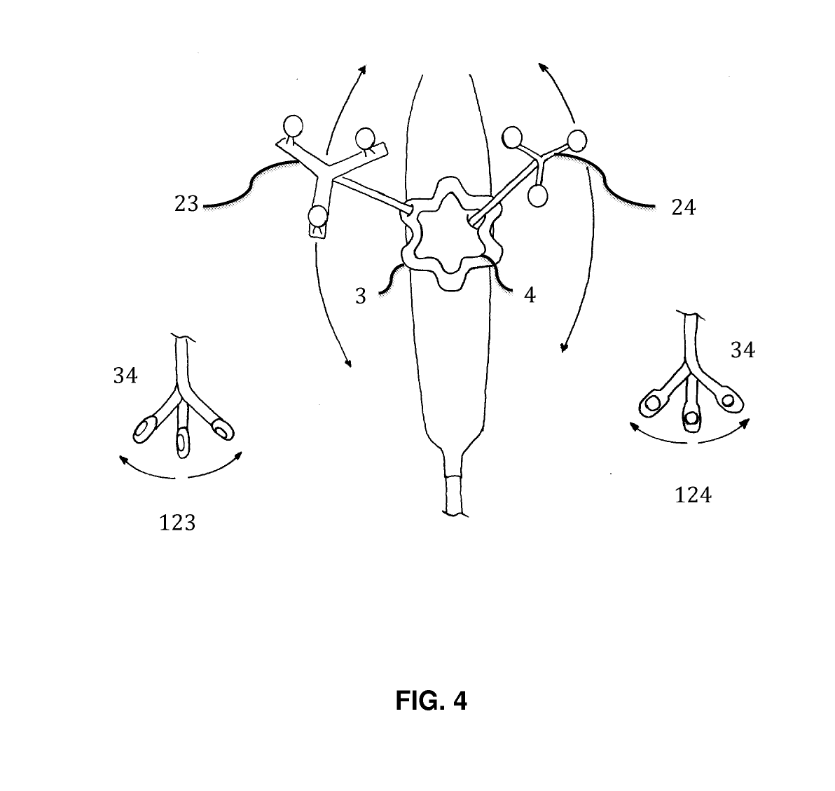

The shaft 27 of TEE probe 10 is seen in FIG. 3A as inserted into the patient's esophagus 33 and the distal tip of the probe 10 where the piezoelectric transducer 34 is located is shown adjacent to the heart. The echocardiographic image 35 as seen in FIG. 3B may be obtained with the transducer located at the above-described position. Various measurements may be performed using the image 35 including identification of major cardiac structures such as a “mitral valve commissure” 36.

The initial position of the piezoelectric TEE transducer 34 may be taken as a reference point for the optical tracking system 1 of the invention according to the location of the optical markers 22, 23 and 24 in FIG. 1. After establishing spatial coordinates the first reference point, ongoing motion of the tip may be tracked using the motion of the external markers 22-24—which in turn may allow detection of secondary reference points and spatial tracking of data acquired from the echocardiographic imaging as seen in FIG. 3B.

The piezoelectric transducer 34 may be configured to emit an ultrasound beam and detect its reflections. The position and orientation of this component can be changed with manipulation of the handle 21 or proximal shaft of the transducer 27 by advancing, withdrawing, turning to left or turning right. These manipulations will be tracked by the navigation system based on the position of the optical marker coupled with handle 21.

The orientation of transducer 34 may be further adjusted using knobs 3 and/or 4 as seen in FIG. 4, which may be located at the handle 21. Movement of the optical marker 23 attached to the knob 3 may be used to detect these manipulations 123. The manipulation of small knob 4 promotes left and right flexion of transducer. Movement of optical marker 24 attached thereto may be used to track these manipulations 124. Information acquired with multi-plane and real time 3D probes can be used to enhance the image from the navigation system.

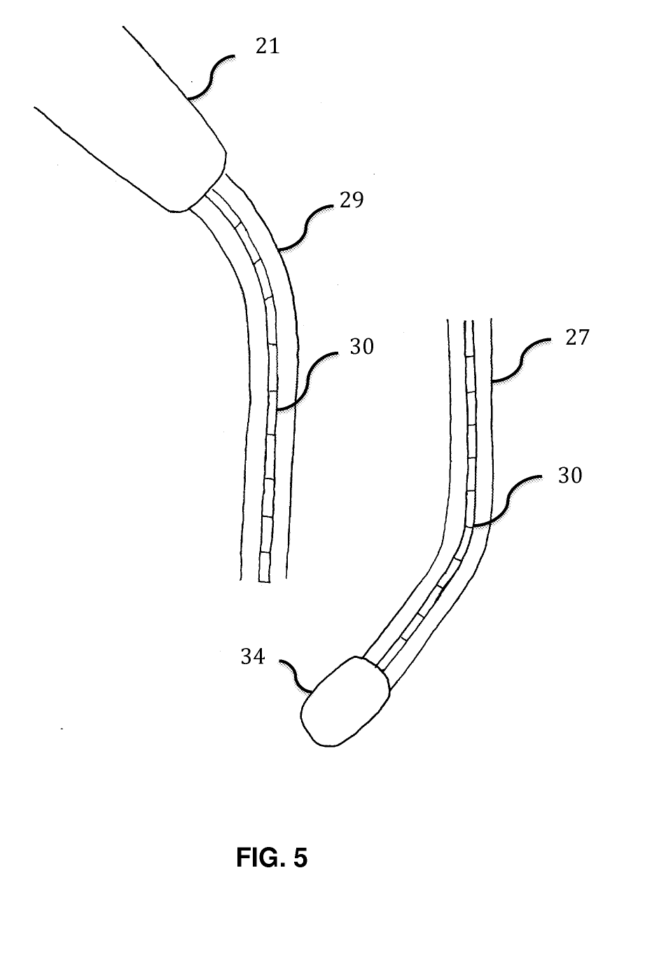

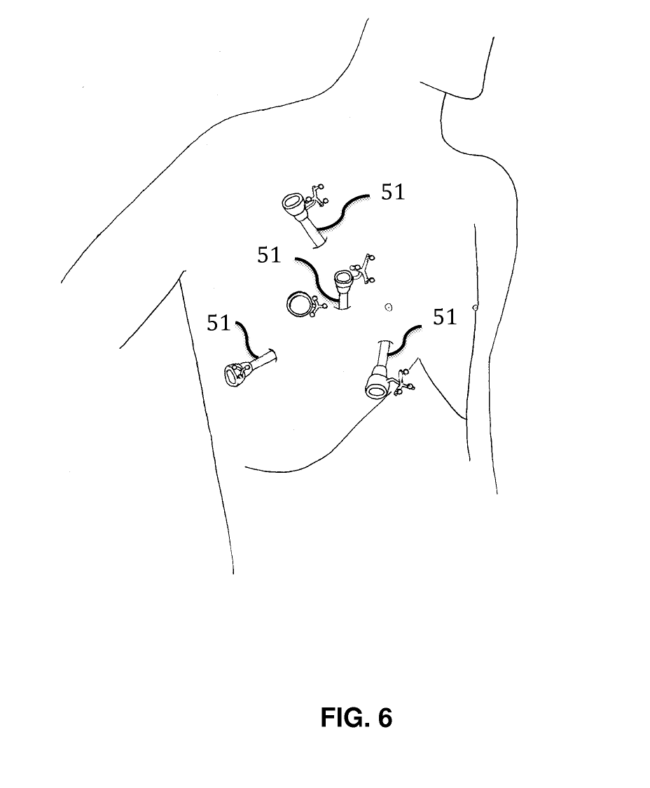

TEE probe 10 deformation may be encountered during positioning in esophagus 33, which may impose certain curvatures on the proximal shaft 29 and middle shaft 27 of transducer 34 that cannot be easily detected simply by movement of the optical markers 22-24. A semi-flexible stylet 30 may also be included with the probe 10 as seen in FIG. 5. The stylet 30 may be comprised of several smaller suitable units for measuring the deformation of the scope. The curves and positional changes not detected by the optical markers 22-24 can be measured by a scope tracking system. Strain gage, electric resistance, accelerometers, gyroscope, and electromagnetic technology can be used for this purpose. The tip of the scope can also be located using site emission sensors to detect electromagnetic signals such as radio waves. These waves can be detected from out side of the patient providing reference from the positioning of the scope.

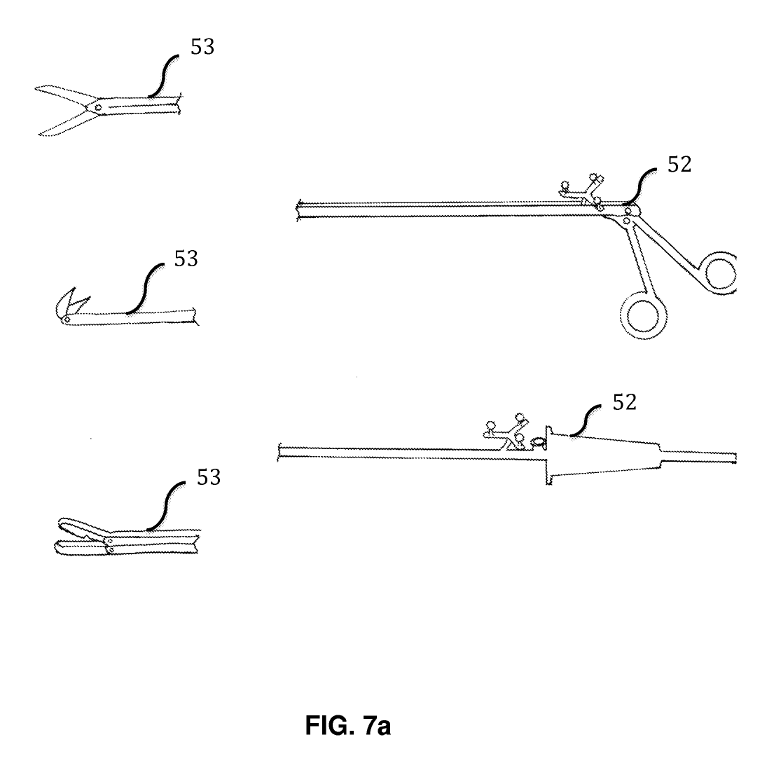

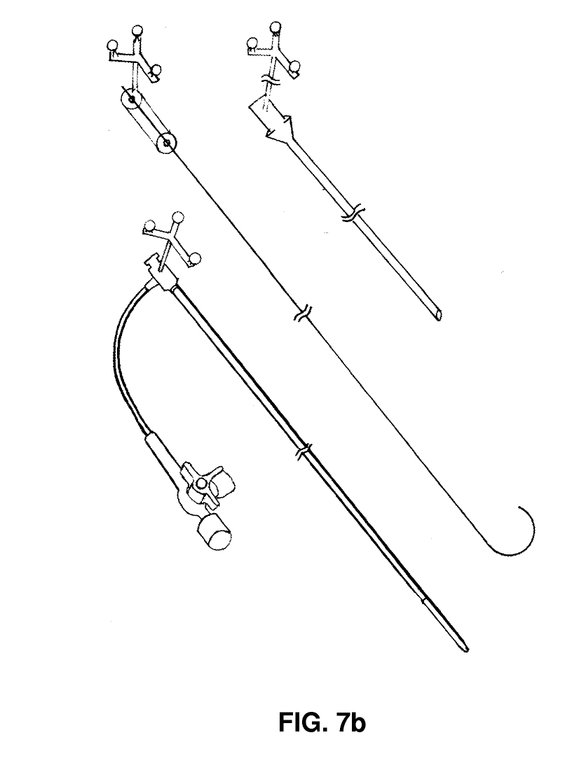

After the acquisition of the reference points, the surgical instruments with optical markers may be oriented using the real time 3D rendered images on display 2. As an example of the surgical instruments with the optical markers, several trocars 51 are seen in FIG. 6 as inserted in a patient's chest. Other types of surgical instruments are illustrated in FIG. 7A. The instrument may have one or more external optical marker positioned on the handle 52 thereof. Such instrument may have various distal ends 53 suitable for various surgical procedures. Those instruments may be strategically inserted and precisely manipulated to carry out the surgical procedure while their representative image may be projected onto image 35 and combined with the patient image for better spatial guidance.

FIG. 7B shows some examples of various needles, catheters, guide wires, sheaths, trocars, and biopsy devices that can be used for the diverse purposes of minimally invasive surgeries and diagnostic procedures. External optical markers may be attached to these devices and electromagnetic stylet may be optionally inserted therein to facilitate their position tracking using the navigation system of the present invention.

In certain aspects the image-guidance system can be configured to project onto image 36 the outline of the respective surgical tools 52 and their distal ends 53. The software can be configured to project their trajectories in the field of view or to plan for their optimal manipulation using advanced spatial resolution of the system as compared with simple human observation techniques.

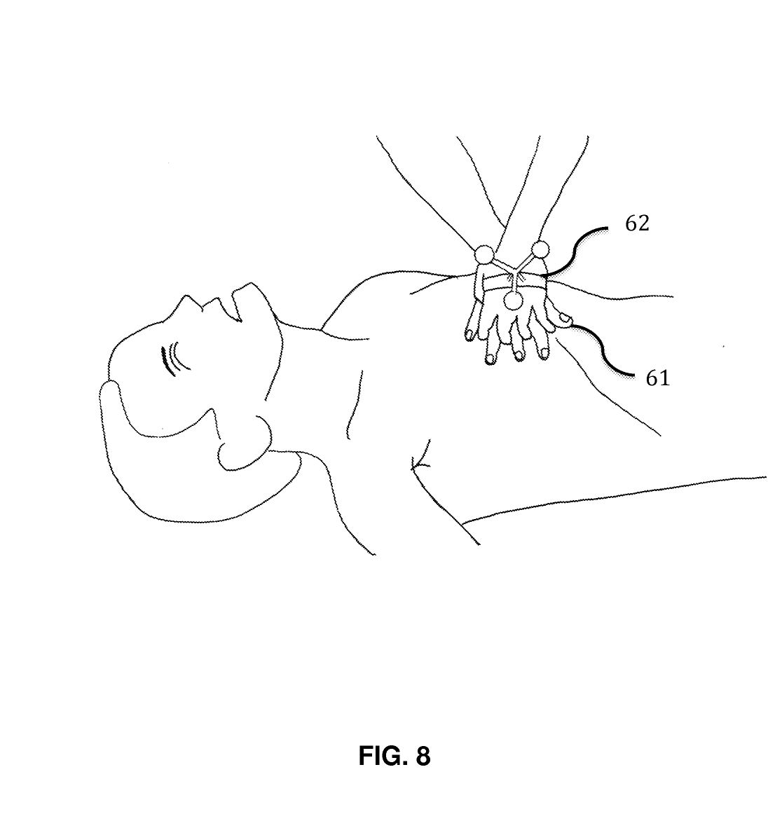

In order to provide more efficient CPR on the area on the patient's chest where compressions should be performed, the system of the invention may be used to represent real time 3D rendered images on display 2. As seen in FIG. 8, the navigation system may provide orientation and special positioning of hands 61 by using an optical marker 62 attached with a hand strap.

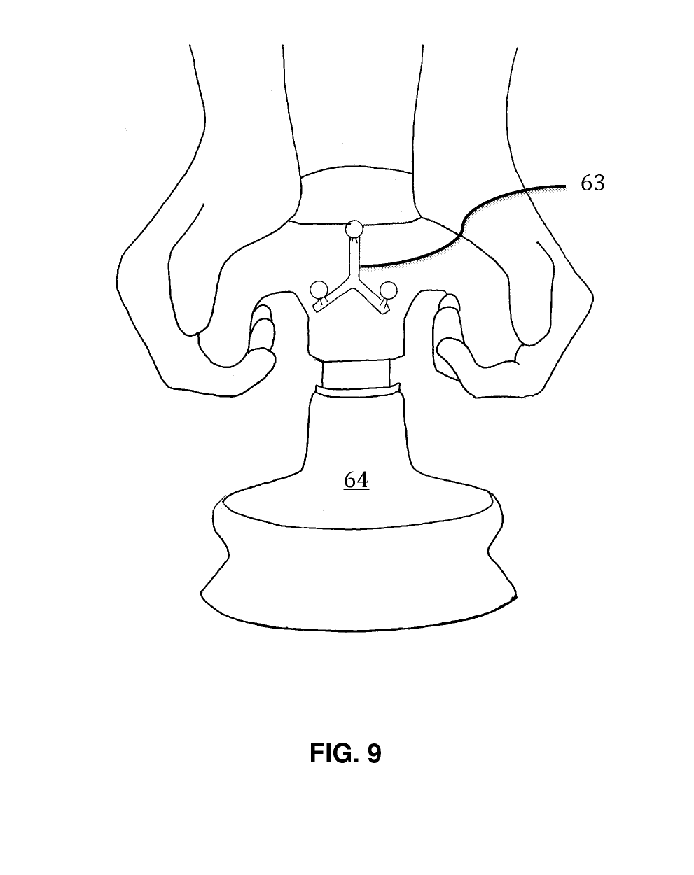

Chest compression can also be mechanically performed by plunger-like devices and the navigation system in this case may be used to direct the area where compressions should be performed. A manually-conducted mechanical CPR device 64 seen in FIG. 9 may be equipped with optical marker 63.

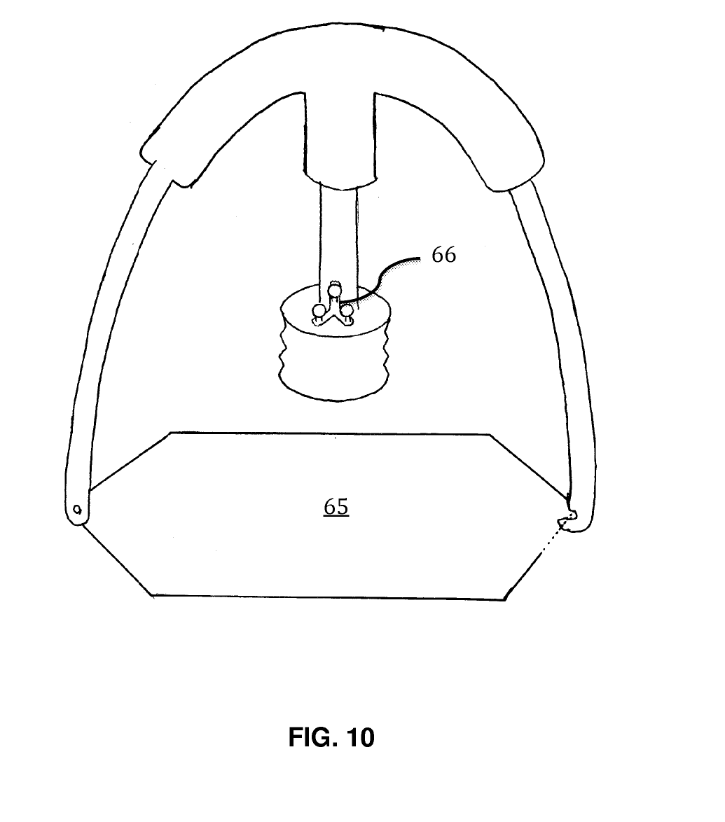

Another device for mechanical CPR 65, which may be mounted around the patient's chest, is seen in FIG. 10. It may be equipped with external optical marker 66 for the same purpose as described above, namely that the area on the chest where the compression should be performed can be dynamically adjusted under direct visualization of the heart structures. In certain aspects the position of the hands or implement used for CPR can be integrated with images of the cardiovascular system showing the effects of compression on the cardiovascular system.

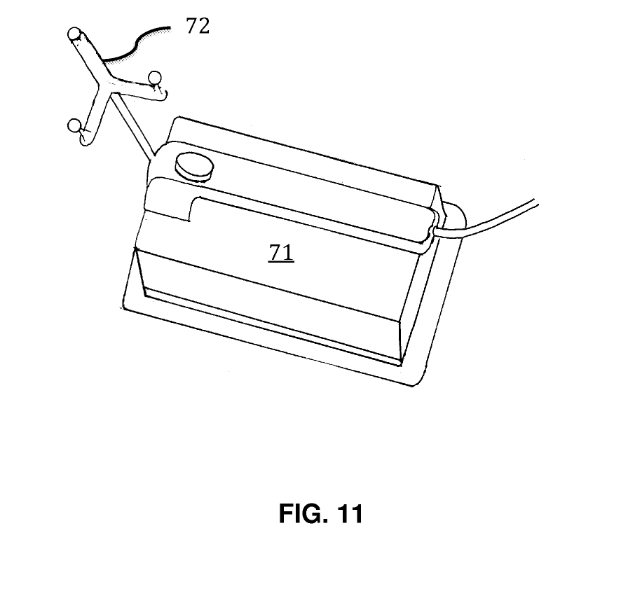

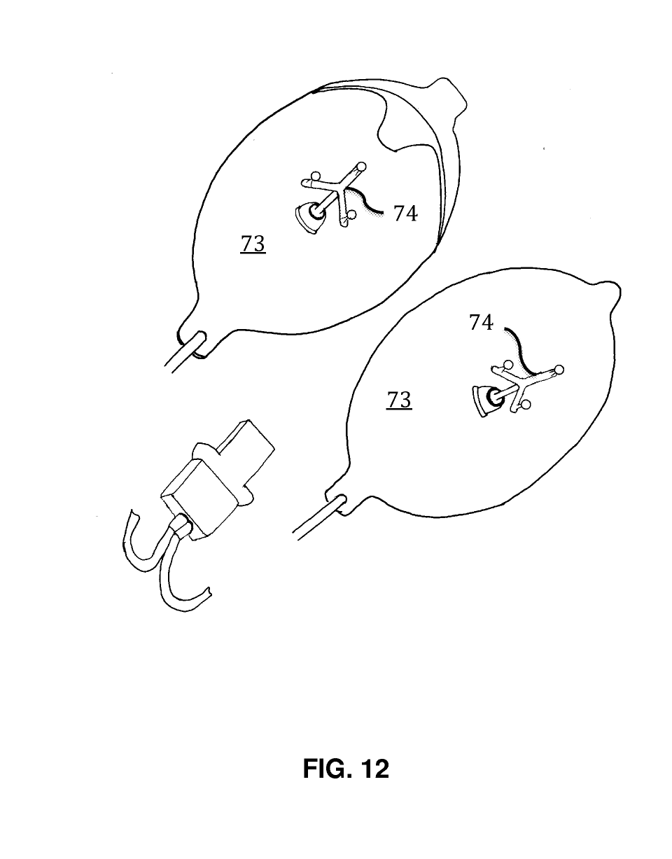

The navigation system of the invention may also provide orientation about the areas on the chest where defibrillating or pacemaker pads may be directed for better oriented delivery of the electric current. Tracking defibrillation handle 71 with optical markers 72 is seen in FIG. 11. Adhesive and disposable defibrillation pads 73 for transcutaneous pacing and for defibrillation may be equipped with respective optical markers 74 as seen in FIG. 12.

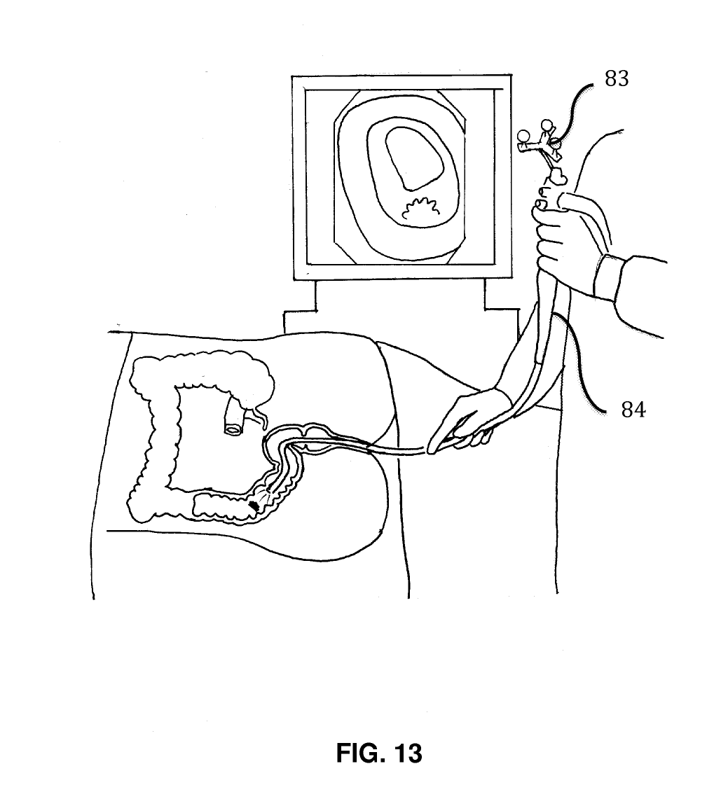

In certain aspects the devices or system described herein can be used in areas other than the torso. In particular, the invention may be used during various laparoscopic and endoscopic procedures. FIG. 13 shows an example of a generic colonoscope-based procedure in which the colonoscope handle 84 may be equipped with the external optical markers 83 so as to assist in image guidance received from the tip of the colonoscope during the procedure.

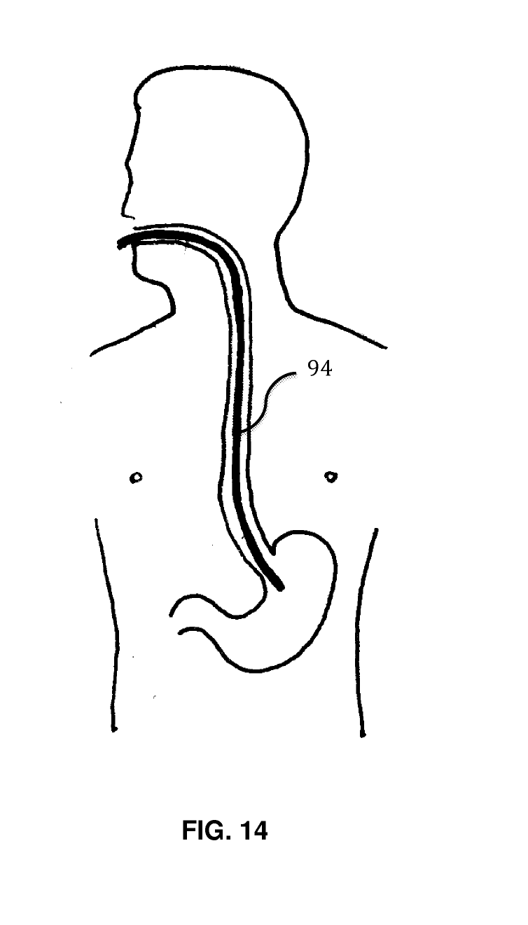

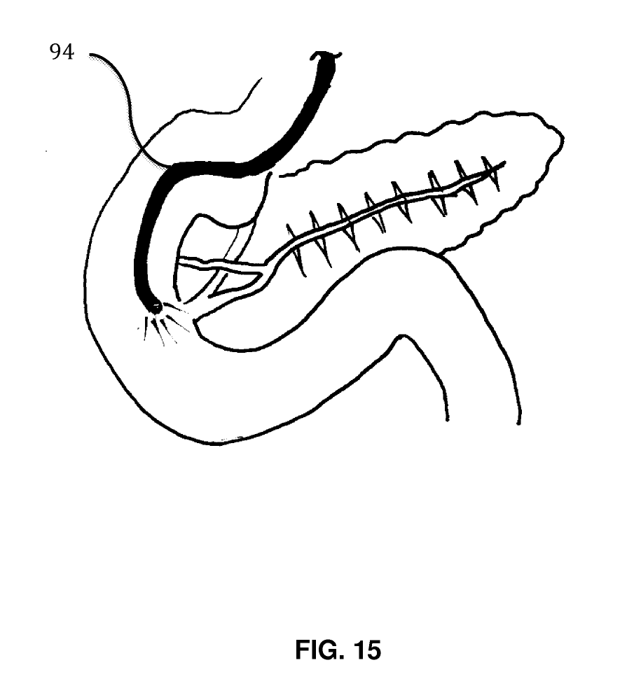

Similarly, FIGS. 14 and 15 illustrate an endoscopic procedure in which the proximal handle of endoscope 94 may be equipped with optical markers—not shown.

In addition to providing spatial coordinates of the handle of the imaging probe by using optical tracking markers, other technologies indicating spatial coordinates may be used for the purposes of the invention, for example gyroscope-based 3D accelerometers. Such devices may be attached to the handle and configured to transmit their real time spatial coordinates to the system of the invention, using wired or wireless modes of data transmission.

1. An image-guided endo-navigation and position tracking system comprising:
an imaging probe configured for insertion into a subject, said imaging probe having a proximal end and a distal end, the imaging probe configured for positioning at a region of interest inside a subject and acquiring in real time image data for the region of interest,
a first optical marker couple to the proximal end of said imaging probe, the first optical marker being configured to remain outside said subject when the imaging probe is inserted in the subject,
a position tracking system comprising two video cameras configured for observing an initial location of said first optical marker and for observing movement of the first optical marker from said initial location, and
a navigation computer system operably connected to said position tracking system and configured to determine initial and ongoing spatial coordinates of said first optical marker, said computer system is further configured to determine initial and ongoing spatial coordinates of the distal end of the imaging probe using the spatial coordinates of the first optical marker, whereby said navigation computer system is configured to determine the initial and ongoing spatial orientation and position of the image data acquired at said distal end of said imaging probe.

2. The system as in claim 1, further comprising a second optical marker coupled to a surgical instrument, whereby said navigation computer system is configured to track spatial orientation and position of said image data and a distal end of said surgical instrument.

3. The system as in claim 2, wherein said navigation system is configured to combine said image data and a projection of said distal end of said surgical instrument and present thereof for visualization on a display.

4. The system as in claim 1, further comprising an electromagnetic stylet sized to be inserted into the imaging probe to reach the distal end thereof, said electromagnetic stylet configured for detecting position of the distal end of the imaging probe regardless a curvature imparted thereto, said navigation system is further configured to read spatial coordinates of said electromagnetic stylet.

5. The system as in claim 1, wherein said imaging probe is selected from a group consisting of a transesophageal echo probe, an endoscope, and a colonoscope.

6. A method of navigation guidance for a surgical procedure or cardio-pulmonary resuscitation, the method comprising the steps of:
inserting an imaging probe into a subject, positioning a distal end of the imaging probe adjacent to and acquiring image data of a region of interest inside the subject, observing a first optical marker arising from a proximal end of the imaging probe, determining initial and ongoing spatial coordinates of the proximal end of the imaging probe
using observations of the first optical marker from step (b), determining initial and ongoing spatial orientation and position of the distal end of the
imaging probe from spatial coordinates of the proximal end thereof, and determining initial and ongoing spatial position and orientation of the image data acquired for the region of interest inside the subject.

7. The method as in claim 6, further including a step of observing initial and ongoing spatial coordinates of a second optical marker arising from a surgical instrument during a surgical procedure or a region of compression during cardiopulmonary resuscitation.

8. The method as in claim 7, where in said second optical marker is attached to a device selected from a group consisting of a hand strap, a mechanical resuscitation device, and a defibrillation device.
